(19) United States
(12) Patent Application Publication
(10) Pub. No.: US 2023/0000892 A1
(43) Pub. Date: Jan. 5, 2023

(54) Solution, Lotion, Gel, and Cream for Use to Prevent Razor Bumps

(71) Applicant: Kenneth Okafor, Eagan, MN (US)

(72) Inventor: Kenneth Okafor, Eagan, MN (US)

(21) Appl. No.: 17/364,790

(22) Filed: Jun. 30, 2021

(51) Int. Cl. A61K 31/7056; A61K 9/06

(52) U.S. Cl. CPC A61K 31/7056; A61K 9/06

(57) ABSTRACT

An article of manufacture for providing a solution, lotion, gel, and cream for use as an aftershave to prevent razor bumps is disclosed. A solution, lotion, gel, and cream having a concentration of clindamycin phosphate between 0.5% and 1.0% may be applied immediately after shaving to a user's skin to prevent razor bumps on various parts of the body that are typically shaved. The present invention may be applied as a skin treatment to the face and neck, underarms, legs, and pubic areas.

### TECHNICAL FIELD

This application relates in general to an article of manufacture for providing a healthy skin treatment, and more specifically, to an article of manufacture providing a post-shaving treatment for the reduction of razor bumps (Pseudofolliculitis)

### BACKGROUND

Most available aftershaves are astringent formulations intended to tighten the skin after shaving. However, research into the pathogenesis of razor bumps shows the involvement of skin microflora, e.g., Staph and Strep sps. Reducing the level of these microflora by applying an antibiotic-based aftershave right after shaving presents a new and unique opportunity to address this problem.

Therefore, a need exists for an article of manufacture for providing a post-shaving treatment for the reduction of razor bumps. The present invention attempts to address the limitations and deficiencies in prior solutions according to the principles and example embodiments disclosed herein.

### SUMMARY

In accordance with the present invention, the above and other problems are solved by providing an article of manufacture for a post-shaving treatment for the reduction of razor bumps according to the principles and example embodiments disclosed herein.

In one embodiment, the present invention is an article of manufacture for providing a solution, lotion, gel, cream, in traditional packaging and as a roll-on applicator for use as an after shave to prevent razor bumps.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description.

### DETAILED DESCRIPTION

This application relates in general to an article of manufacture for providing a healthy skin treatment, and more specifically, to an article of manufacture for providing a post-shaving treatment product for the reduction of razor bumps according to the present invention.

Various embodiments of the present invention will be described herein. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two components recited in succession may in fact be executed substantially concurrently or may sometimes be utilized in the reverse order, depending upon the functionality and acts involved.

The terms “individual” and “user” refer to an entity, e.g., a human, using an article of manufacture providing a post-shaving treatment for the reduction of razor bumps associated with the invention. The term user herein refers to one or more users.

The term “invention” or “present invention” refers to the invention being applied for via the patent application with the title “Solution, lotion, Gel, and Cream for Use to Prevent Razor Bumps.” Invention may be used interchangeably with treatment.

In general, the present disclosure relates to an article of manufacture for providing a post-shaving treatment for the reduction of razor bumps according to the present invention. To better understand the present invention, a solution, lotion, gel, and cream of 0.75% to 1% clindamycin phosphate may be used on a user's skin after shaving to prevent shaving bumps on the face, legs, and pubic areas.

Staphylococcus aureus is a gram-positive, round-shaped bacterium, frequently found on the skin. Staph aureus is a member of the firmicutes phylum of bacteria and is a usual member of the microbiota of the body. Although staph aureus usually acts symbiotically as a human microbiota, it can also become an opportunistic pathogen that is a common cause of skin infections including abscesses and razor bumps. The susceptibility of Staphylococcus and Propionibacterium acnes to clindamycin is the basis of the present invention in which clindamycin could be an effective after shave option.

Clindamycin phosphate is an off patent (generic) prescription product available as a 1% solution, lotion, pledgets and gel for treatment of acne. Some embodiments of these therapeutic dosage forms may be reformulated as cosmetics for use as aftershaves with an addition of perfumes, allantoin, aloe vera, and other moisturizers and made available in traditional packaging or as a roll-on for easy application to prevent razor bumps. The present invention may be applied immediately after shaving for effective use to the face and neck, underarms, legs, and pubic areas. The male and female versions may be perfumed differently; for example an unscented, a sandalwood version for men, and a jasmine version for women. Many additional perfumes may be used with the present invention.

Clindamycin is currently available (by prescription only) as a 1% solution, gel, lotion, or suspension, pledgets and in combination with benzoyl peroxide for the treatment of acnes. The available gel is thick and sticky by design. the solution is too drying due to a high alcoholic content and the lotion lacks effective moisturizers because of the intended purpose-acne treatment. The after shave, in addition to being scented, will be reformulated in accordance with the pharmaceutical elegance of typical aftershave gels and lotions. Typically, a lower concentration of the inactive gel forming hydrocolloids, Ph adjustments and other inactive ingredients adjustments will produce a less sticky gel and a better moisturizing lotion, producing products that resemble the consistency and feel of typical aftershaves. A 1% concentration of clindamycin within the gel is preferrable, although a lower concentration down to at least 0.75%, is sufficiently adequate for the purpose intended. The lower concentration thereotically increases the chance of bacteria resistance. The lower concentration also is also within the scope of the present invention with the slightly reduced but sufficient efficacy as noted above.

To determine the above results, available acne gels, creams, suspension, and lotions were used as proof of concept in a small sample size and the results showed the treatment was approximately 100% effective in preventing razor bumps when used immediately after shaving. The testing was performed using a concentration of 0.5% and 0.75% and 1% with ALL concentrations demonstrating corresponding effective results.

Even though particular combinations of features are recited in the present application, these combinations are not intended to limit the disclosure of the invention. In fact, many of these features may be combined in ways not specifically recited in this application. In other words, any of the features mentioned in this application may be included in this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed is:

1. A solution for use as an aftershave to prevent razor bumps, the solution comprises 1% clindamycin phosphate solution.

2. A lotion for use as an aftershave to prevent razor bumps, the suspension comprises 1% clindamycin phosphate lotion.

3. A gel for use as an aftershave to prevent razor bumps, the gel comprises 1% clindamycin phosphate gel.

4. A cream for use as an aftershave to prevent razor bumps, the cream comprises 1% clindamycin phosphate cream.
